(19) United States
(12) Patent Application Publication
(10) Pub. No.: US 2010/0016756 A1
(43) Pub. Date: Jan. 21, 2010

(54) PRESSURE SENSOR OPERATING IN A FLUID MEDIUM

(76) Inventor: Eliav TAHAR, Tel Aviv (IL)

Correspondence Address: APPELFELD ZER FISHER, B.S.R Tower 1, Floor 16., 2 Ben Gurion Road, Ramat Gan, 52573, IL

(21) Appl. No.: 12/176,378

(22) Filed: Jul. 20, 2008

(51) Int. Cl. A61B 5/03

(52) U.S. Cl. 600/561

(57) ABSTRACT

A pressure sensor and a method of measuring pressure signals in a fluid medium. The pressure sensor comprises a sensing element for transducing pressure signals to electric signals and a mechanical amplifier connected to the sensing element, comprising an immersed appendage and a transmission element. The sensing element is enclosed within a case, and the mechanical amplifier is structured to seal the case and isolate the sensing element from the fluid medium. Pressure signals in the fluid medium cause movements in the appendage that are transmitted via the elongated transmission element to the sensing element that is isolated from the fluid medium. The method comprises the stages: Sensing the pressure signal and transmitting it via a transmission element to a piezoelectric sensing element inside the measuring apparatus.

### FIELD OF THE INVENTION

The present invention generally relates to the field of sensing. More particularly, the present invention relates to a pressure sensor.

### BACKGROUND OF THE INVENTION

WO2005112615, which is incorporated herein by reference in its entirety, discloses a bolus configured to process an overall acoustic signal emanated from different signal sources within the animal, and output values indicative of respective physiological parameters of the animal indicative of its health condition, such as heartbeat rate, respiration rate, rumination activity, etc.

U.S. 61/044,517 which is incorporated herein by reference in its entirety, discloses a system and method for monitoring animal health utilizing capsules traveling the digestive tract from mouth to rectum. U.S. 61/044,514 which is incorporated herein by reference in its entirety, discloses a system and method for monitoring physiological conditions using continuous telemetric measurement of physiological parameters.

### SUMMARY OF THE INVENTION

The present invention discloses a pressure sensor operating in a fluid medium. The sensor comprises a sensing element for transducing a pressure signal to an electric signal and a mechanical amplifier operatively connected to the sensing element. The sensing element is enclosed within a case, and the mechanical amplifier is structured to seal the case and isolate the sensing element from the fluid medium. The mechanical amplifier comprises an appendage connected externally to the sealed case and immersed in the fluid medium, and an elongated transmission element operatively connecting the appendage to the sensing element in such a way that the pressure signal is transmitted from the appendage to the sensing element. Pressure signals in the fluid medium cause movements in the appendage that are transmitted via the elongated transmission element to the sensing element that is isolated from the fluid medium.

In embodiments, the sensing elements comprises an upper plate connected to the elongated transmission element and a piezoelectric plate connected to the upper plate, wherein the upper plate with the elongated transmission element are arranged to amplify the pressure signal to allow an optimal deformation of the piezoelectric plate.

In embodiments, the appendage is an integral part of the case and comprises an inner notch. The distal end of the elongated transmission element is arranged to fit in this inner notch, such that movements of the appendage are transmitted to the elongated transmission element.

The present invention further discloses a pressure sensor operating in a fluid medium. The sensor comprises a sensing element for transducing a pressure signal to an electric signal, that is enclosed within a sealed case as well as an elongated transmission element operatively connected to the sensing element and to the inner side of the sealed case, in such a way that the pressure signal is transmitted from the surface of the sealed case to the sensing element. The pressure signals in the fluid medium cause movements in the surface of the sealed case that are transmitted via the elongated transmission element to the sensing element. In embodiments, the front edge of the sealed case has a thin and flexible side walls and a thicker central wall.

The present invention further discloses a method of measuring pressure signals in a fluid medium. The method comprises the stages: (i) sensing a pressure signal in a fluid medium by a sealed case of a measuring apparatus, (ii) transmitting the pressure signal from the fluid medium to a transmission element inside the measuring apparatus, (iii) transmitting the pressure signal from the transmission element to a piezoelectric sensing element, and (iv) transducing the pressure signal into an electric signal by the piezoelectric sensing element.

In embodiments, the method further comprises connecting at least one appendage to the sealed case, wherein the appendage improves the sensing of a pressure signal; and transmitting the pressure signal from the fluid medium into a measuring apparatus while maintaining the measuring apparatus sealed against the fluid medium.

### BRIEF DESCRIPTION OF THE DRAWINGS

The subject matter regarded as the invention will become more clearly understood in light of the ensuing description of embodiments herein, given by way of example and for purposes of illustrative discussion of the present invention only, with reference to the accompanying drawings (Figures, or simply “FIGS.”), wherein:

FIGS. 1A, 1B, 1C are block diagrams illustrating apparatuses for monitoring the physiological condition of animals located within the animals digestive tract. FIG. 1A illustrates an apparatus according to prior art, while FIGS. 1B and 1C illustrate apparatuses according to some embodiments of the invention.

FIG. 2 is an illustration of a configuration of a sensing element, in side view and in top view, according to some embodiments of the invention.

FIG. 3 is an illustration of an elongated transmission element connected to a sensing element, in perspective and in side view, according to some embodiments of the invention.

FIG. 3A is an illustration of a sensing element, in side view, according to some embodiments of the invention.

FIG. 4 is a cross section of a pressure sensor operating in a fluid medium, according to some embodiments of the invention.

FIG. 5 is a cross section of a pressure sensor operating in a fluid medium, according to some embodiments of the invention.

FIG. 6 is a cross section of a pressure sensor operating in a fluid medium, according to some embodiments of the invention.

FIG. 7 is a cross section of a pressure sensor operating in a fluid medium, according to some embodiments of the invention.

FIG. 8 is a cross section of a pressure sensor operating in a fluid medium, according to some embodiments of the invention.

FIG. 9 is a flowchart illustrating a method for measuring pressure signals in a fluid medium, according to some embodiments of the invention.

### DETAILED DESCRIPTIONS OF SOME EMBODIMENTS OF THE INVENTION

The present invention discloses a pressure sensor operating in a fluid medium and a method for measuring pressure signals in a fluid medium. The disclosure comprises different embodiments of a sensing element and of a mechanical amplifier, arranged to transmit the pressure signal from the external fluid medium to a sensing element inside of sealed case.

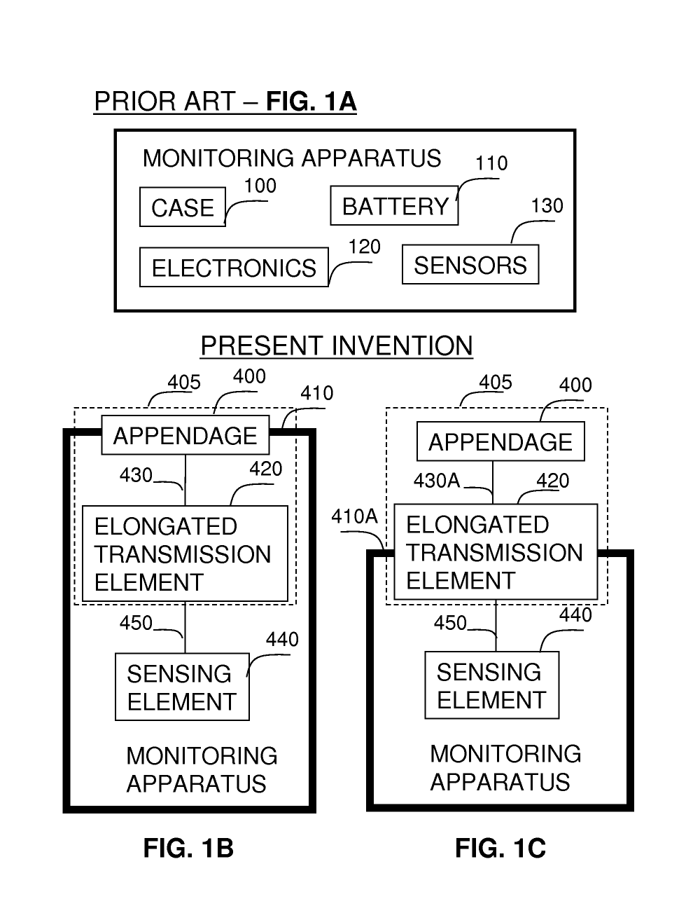

FIGS. 1A, 1B, 1C are block diagrams illustrating apparatuses for monitoring the physiological condition of animals located within the animals digestive tract. FIG. 1A illustrates an apparatus according to prior art, while FIGS. 1B and 1C illustrate apparatuses according to some embodiments of the invention. The apparatus according to prior art comprises a case 100 holding a battery 110 and electronic elements 120, as well as different sensors 130. The apparatus according to some embodiments of the invention comprises a pressure sensor operating in a fluid medium. The sensor comprises a sensing element 440 and a mechanical amplifier 405 comprising an appendage 400 and an elongated transmission element 420. The appendage 400 is connected to the elongated transmission element 420 by connecting means 430 and the elongated transmission element 420 is connected to the sensing element 440 by connecting means 450. The mechanical amplifier 405 is connected to the case 410 of the monitoring apparatus either (i) via the appendage 400 (FIG. 1B), i.e. the appendage 400 is connected to the case 410 directly or is an integral part of the case 410 (e.g. cast together with the case 410), while the connecting means 430 is internal to the case 410, or (ii) via the elongated transmission element 420 (FIG. 1C) i.e. the elongated transmission element 420 is connected to the case 410A and the connecting means 430 is external to the case 410A. In the latter case the appendage 400 is not part of the case 410A. The appendage 400 is immersed in said fluid medium and transmits pressure signals via connecting means 430 to the elongated transmission element 420. Together they build the mechanical amplifier 405 which is operatively connected to the sensing element 440 and structured to seal the case 410 and isolate the sensing element 440 from the fluid medium, as well as adjust the intensity of pressure signal reaching the sensing element 440. The connecting means 450 allows the pressure signal to be transmitted to the sensing element 440 without damaging it, and the sensing element 440 transduces the pressure signal to an electric signal. Altogether—pressure signals in the fluid medium cause movements in the appendage 400 that are transmitted via the elongated transmission element 420 to the sensing element 440 that is kept isolated from the fluid medium.

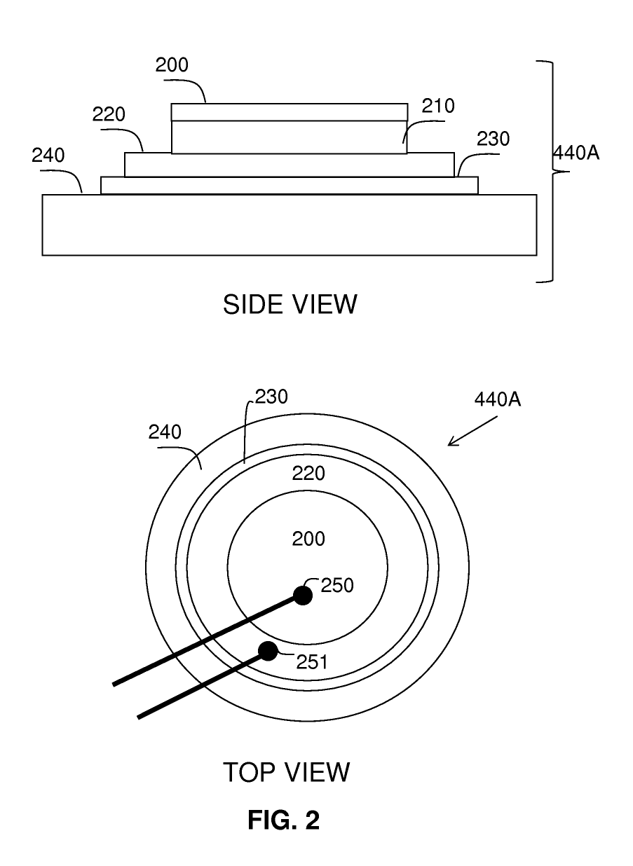

FIG. 2 is an illustration of a configuration of a sensing element 440A, in side view and in top view, according to some embodiments of the invention. The sensing element 440A comprises a piezoelectric ceramic chip 210 with a metal coating 200, attached upon a thin metal plate 220 (e.g. made of brass). A first electric contact 250 is connected to the metal coating 200 of piezoelectric ceramic chip 210, and a second electric contact 251 is connected to the thin metal plate 220. These plates 200, 210, 220 are attached upon a ceramic isolation plate 230 (e.g. over 30 MΩ), that is attached upon a base metal plate 240 (e.g. made of brass). The base metal plate 240 supports the other plates. According to some embodiments of the invention, all plates are between 0.1 mm and 0.3 mm thick, and between 20 mm and 40 mm in diameter. The sensing element 440A is an embodiment of the sensing element 440.

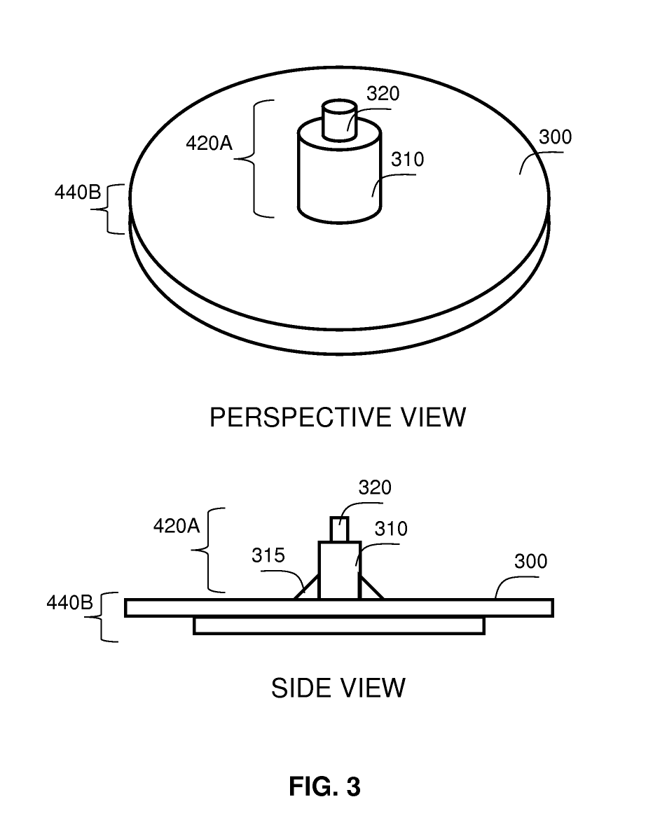

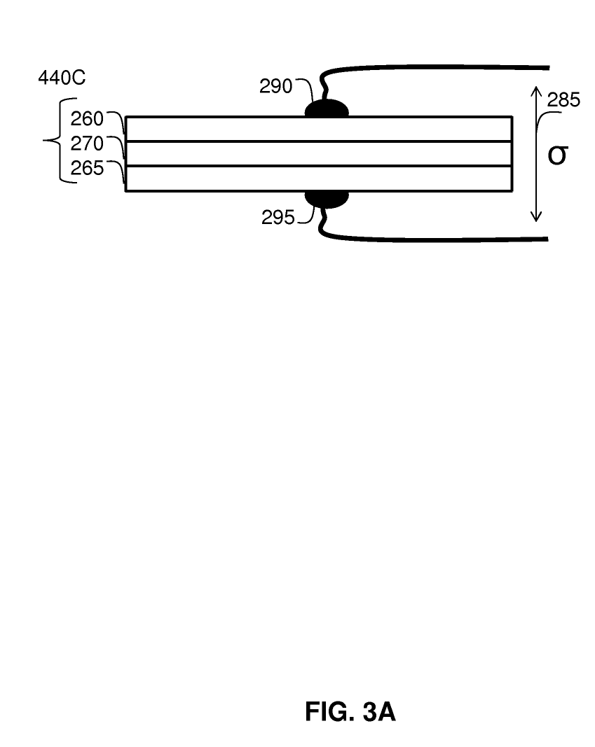

FIG. 3 is an illustration of an elongated transmission element 420A connected to a sensing element 440B, in perspective and in side view, according to some embodiments of the invention. The sensing element 440B is an embodiment of the sensing element 440. The elongated transmission element 420A is an embodiment of the elongated transmission element 420, and may comprise a main subunit 310 and a secondary subunit 320 which enables connecting the elongated transmission element 420A to the appendage 400 (as part of an embodiment of the connecting means 430). The main subunit 310, which may be a pin, is connected to the middle of the sensing element 440B, which may be a plate. The connection may be e.g. by soldering, leaving some solder 315 as support. The soldering is an embodiment of the connecting means 450.

The sensing element 440B may comprise an upper plate 300 and a lower ceramic, piezoelectric plate 305 connected to the upper plate 300. The sensing element 440B may comprise additional layers. The structure of the sensing element 440B is such that it allows an optimal deformation of a piezoelectric element—i.e. large enough to be measured and permit good resolution of the measurement data, yet small enough to avoid damage to the piezoelectric element. In addition, the structure of the sensing element 440B must allow connection to the elongated transmission element 420A and of electric contacts without damage to the piezoelectric element. The elongated transmission element 420A transmits a pressure signal received in its distal end 320 to the proximal sensing element 300. Utilizing the lever of the elongated transmission element 420A, the pressure signal may be amplified at the sensing element 440B. The distal end 320 of the elongated transmission element 420A may receive the pressure signal from the case of the apparatus, or from an appendage connected externally to the case.

According to some embodiments of the invention the elongated transmission element 420 may comprise several transmission elements interconnected by joints, which transmit the pressure signal to the sensing element 440 in a range of intensities allowing an optimal analysis of the electric signal to which the pressure signal is transduced.

FIG. 3A is an illustration of a sensing element 440C, in side view, according to some embodiments of the invention. The sensing element 440C comprises an upper plate 260 that may be connected to the elongated transmission element 420A and a piezoelectric plate 270 connected to the upper plate 260. The upper plate 260 is arrange to amplify the pressure signal coming from the elongated transmission element 420 and to allow an optimal deformation of the piezoelectric plate 270, according to its specifications. The sensing element 440C is an embodiment of the sensing element 440.

The sensing element 440C may further comprise a lower metal plate 265 connected to the piezoelectric plate 270 such that at least a portion of the piezoelectric plate 270 is enclosed between the upper plate 260 and the lower metal plate 265, an upper connection 290 to the upper plate 260 and a lower connection 295 to the lower metal plate 265. The conductivity 285 of the piezoelectric plate 270 may be measured between the connections 290 and 295 and be utilized to indicate the mechanical stress applied to the piezoelectric plate 270. Changes in the mechanical stress represent changes in the pressure signal in the fluid medium outside the case.

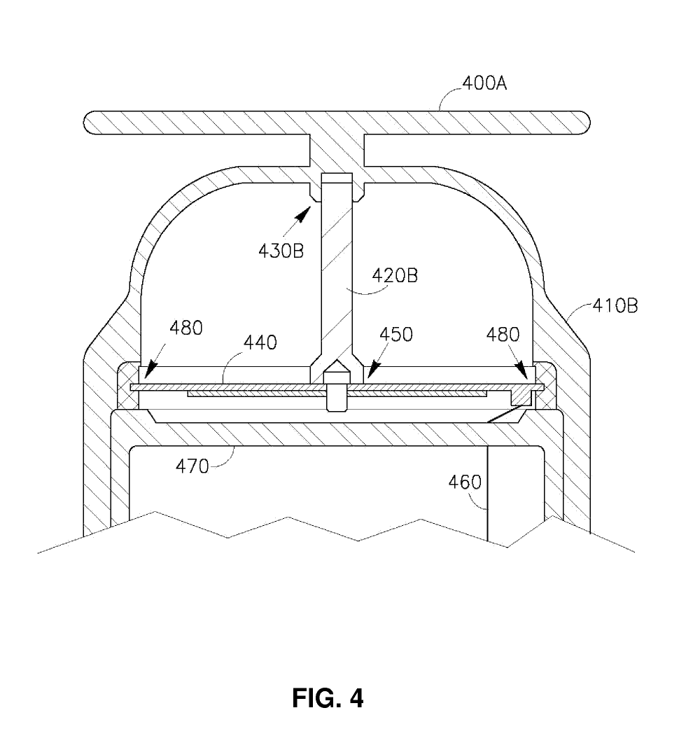

FIG. 4 is a cross section of a pressure sensor operating in a fluid medium, according to some embodiments of the invention. The pressure sensor comprises a sensing element 440 enclosed within a sealed case 410B, and a mechanical amplifier comprising an appendage 400A connected externally to the sealed case 410B and immersed in the fluid medium as well as an elongated transmission element 420B that operatively connects the appendage 400A to the sensing element 440. The appendage 400A may be shaped as a plate. The elongated transmission element 420B is connected to the sensing element 440 by a connection area 450, e.g. by soldering. The connection area 450 may be arranged to allow connecting the elongated transmission element 420B to the sensing element 440 without damaging it, and in a way allowing the transmission of a pressure signal to the sensing element 440. The sensing element 440 is arranged to allow an optimal deformation of a piezoelectric element—i.e. large enough to be measured and permit good resolution of the measurement data, yet small enough to avoid damage to the piezoelectric element. The sensing element 440 may comprise two to five layers—a piezoelectric layer, a metal layer connected to the elongated transmission element 420B and other layers for support and insulation.

According to some embodiments of the invention, pressure signals in the fluid medium move the appendage 400A, which in turn moves the elongated transmission element 420. The elongated transmission element 420B transmits the pressure signal to the sensing element 440, which transduces the pressure signal to an electric signal. The mechanical amplifier thus transfers the mechanical signal from the external medium to the sensing element 440 that is enclosed within the sealed case 410B, and its structure is arranged to deliver typical pressure signals to the sensing element 440 in an optimal intensity, allowing an efficient detection of the pressure signal. The electric signal is transmitted by wires 460 to the electronic circuits enclosed in an inner case 470. The sensing element 440 is attached to the case 410B by to flexible connectors 480, attaching it firmly yet flexible to the inner wall of the case 410B, allowing some movement caused be the mechanical stressed applied to it by the elongated transmission element 420B.

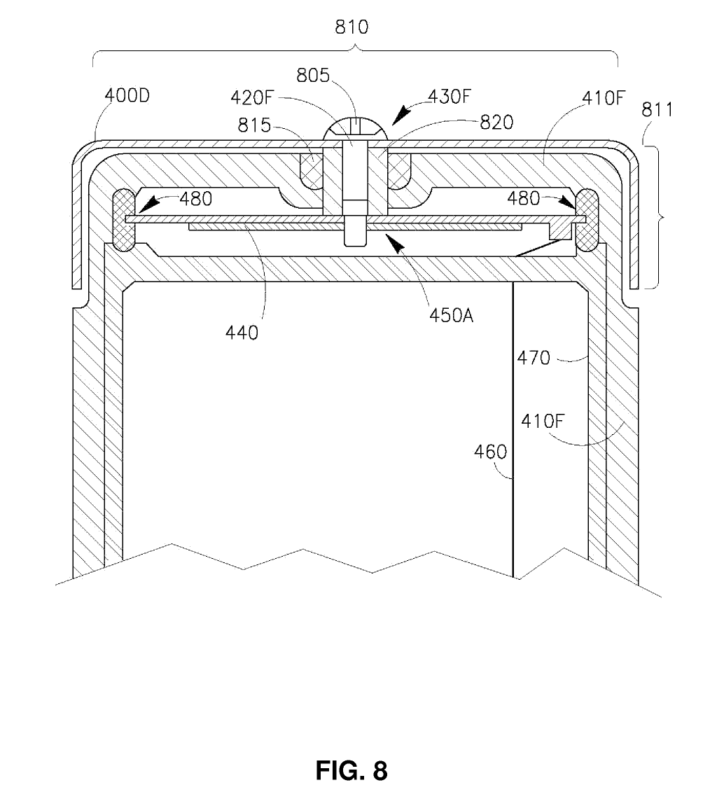

According to some embodiments of the invention, the mechanical amplifier receives and transmits a pressure signal from outside the case 410B to the sensing element 440 within the case. The interface 430B of the appendage 400A and the elongated transmission element 420B is thus both sealed and permits an efficient transmission of the mechanical signal. According to some embodiments, the appendage 400A is connected upon the case 410B and the interface 430B is sealed utilizing e.g. O rings (see FIG. 8). According to other embodiments, the appendage 400A is an integral part of case 410B (e.g. cast together with the case 410B) and comprises an inner notch at its base. The distal end of the elongated transmission element 420 may be arranged to fit into the inner notch, in a way that movements of the appendage 400 are transmitted to the elongated transmission element 420.

Assembling the apparatus, the elongated transmission element 420B is inserted into the notch from within the case 410B. These embodiments allow a better sealing of the case which endures rougher environment. The mechanical amplifier is structured to seal the case 410B and isolate the sensing element 440 and the electronics from the fluid medium. The good sealing in crucial for the sensor to operate in the digestive tract which is characterized by strong movements (digestive contractions) and low pH.

The appendage 400A is an embodiment of the appendage 400, the case 410B is an embodiment of the case 410, the elongated transmission element 420B is an embodiment of the elongated transmission element 420. The interface 430B arises from an embodiment of the connecting means 430.

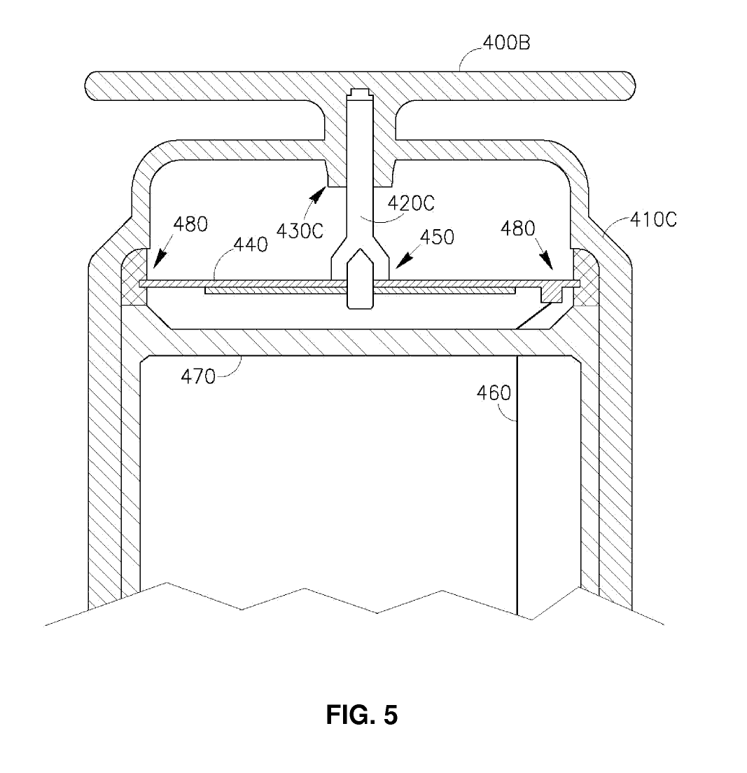

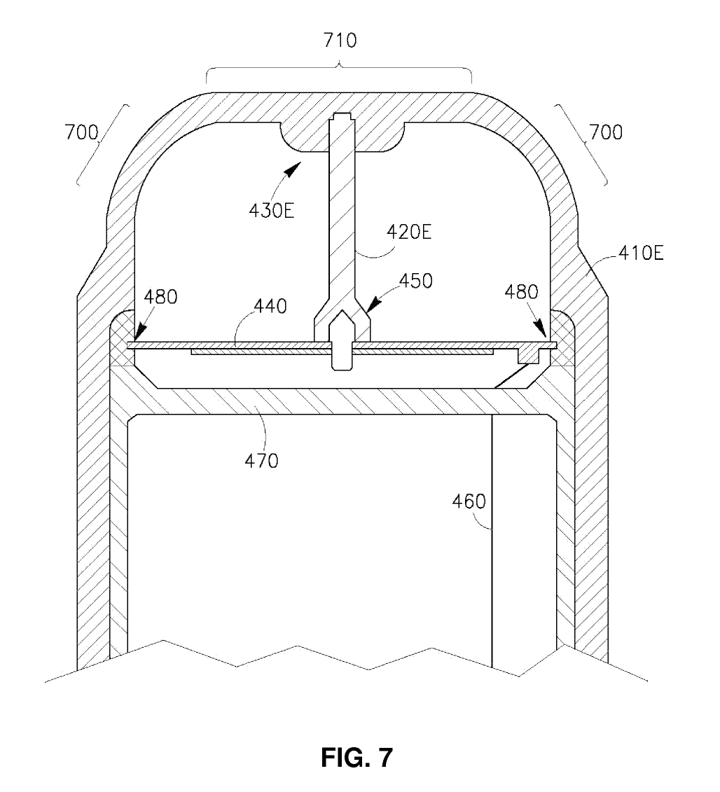

FIG. 5 is a cross section of a pressure sensor operating in a fluid medium, according to some embodiments of the invention. FIG. 5 illustrates an embodiment of the pressure sensor which is similar to the embodiment illustrated in FIG. 4, except for the connection area of the elongated transmission element 420C. In FIG. 5, the interface 430C of the appendage 400B and the elongated transmission element 420C is larger and allows a stronger pressure signal transmission from the appendage 400 to the elongated transmission element 420C. In addition, the flatter form of the front edge of the case 410C increases the proportion of signal coming from the appendage 400B in respect to the proportion of signal coming from the case 410C (compare to FIG. 7 below). The appendage 400B may be shaped as a plate.

According to other embodiments, the appendage 400B is an integral part of case 410C (e.g. cast together with the case 410C) and comprises an inner notch at its base. The distal end of the elongated transmission element 420 may be arranged to fit into the inner notch, in a way that movements of the appendage 400 are transmitted to the elongated transmission element 420. The inner notch and the fitting elongated transmission element 420C are represented in the interface 430C in FIG. 5.

The appendage 400B is an embodiment of the appendage 400, the case 410C is an embodiment of the case 410, the elongated transmission element 420C is an embodiment of the elongated transmission element 420. The interface 430C arises from an embodiment of the connecting means 430.

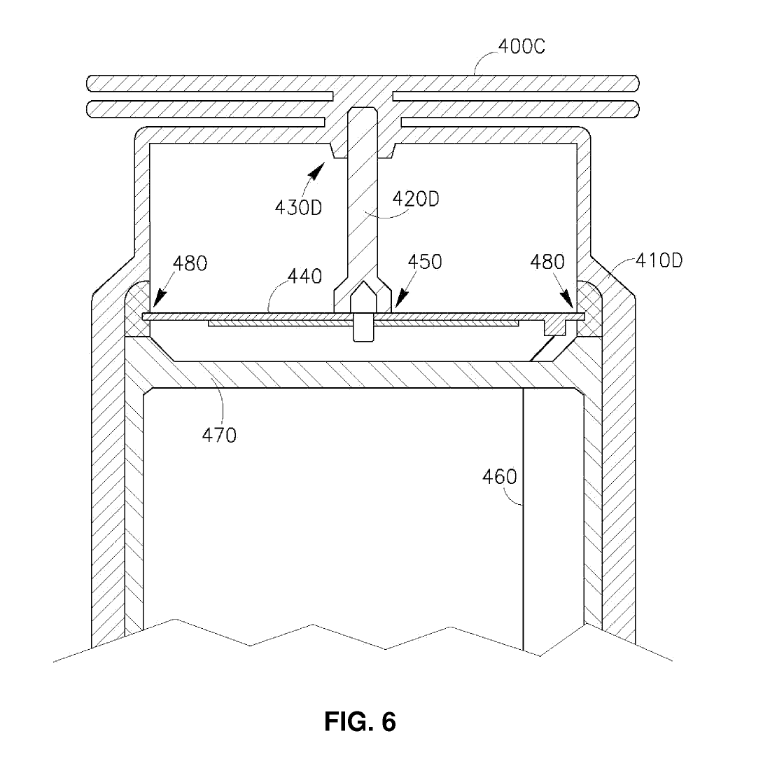

FIG. 6 is a cross section of a pressure sensor operating in a fluid medium, according to some embodiments of the invention. FIG. 6 illustrates an embodiment of the pressure sensor with a two layered appendage 400C allowing a higher sensitivity to pressure signals in the fluid medium. The form of the case 410D and the interface 430D of the appendage 400C with the elongated transmission element 420D are arranged to allow maximal support of the appendage 400C and an optimal pressure signal transduction. The appendage 400C may be shaped as two interconnected parallel plates.

The appendage 400C is an embodiment of the appendage 400, the case 410D is an embodiment of the case 410. The elongated transmission element 420D is an embodiment of the elongated transmission element 420. The interface 430D arises from an embodiment of the connecting means 430.

FIG. 7 is a cross section of a pressure sensor operating in a fluid medium, according to some embodiments of the invention. FIG. 7 illustrates an embodiment similar to the embodiment illustrated in FIG. 4, but lacking the appendage 400. In this embodiment the pressure signal in the fluid medium is transmitted to the elongated transmission element 420E from the front edge of the case 410E alone. The front edge of the case 410E may be shaped to allow an optimal signal receipt and transmission, e.g. dome shaped. The pressure sensor comprises a sensing element 440 and an elongated transmission element 420E. The sensing element 440 transduces a pressure signal to an electric signal and is enclosed within a sealed case 410E. The elongated transmission element 420E is operatively connected to the sensing element 440 and to the inner side of the sealed case 410E, such that the pressure signal is transmitted from the surface of the sealed case 410E to the sensing element 440. The pressure signals in the fluid medium cause movements in the surface of the sealed case 410E, which are transmitted via the elongated transmission element 420E to the sensing element 440. In this embodiment, the appendage 400 is unified with the front end of the sealed case 410E. The front end of the sealed case 410E may be shaped to facilitate the reception of the pressure signal, e.g. have thin and somewhat flexible side walls 700 and thicker central wall 705 that may move and transmit the pressure signal to the elongated transmission element 420E. The interface 430E is arranged to transmit the movements of the front end of the sealed case 410E to the elongated transmission element 420E.

The case 410E is an embodiment of the case 410. The elongated transmission element 420E is an embodiment of the elongated transmission element 420. The interface 430E arises from an embodiment of the connecting means 430.

FIG. 8 is a cross section of a pressure sensor operating in a fluid medium, according to some embodiments of the invention. FIG. 8 illustrates an embodiment of the pressure sensor which differs in the form and connection method of the appendage and transmission element to the case. According to some embodiments of the invention, an appendage 400D is connected upon the case 410F and not cast as part of it. For example, the appendage 400D may be connected by a connector 420F (e.g. a screw 805) going through the case 410F and connecting to the sensing element 440. The connector 420F is thus a part of the elongated transmission element 420, and transmit the pressure signal received by the appendage 400D from the surrounding fluid medium to the sensing element 440. The connector 420F may be supported by a threaded hole 820 connected to the case 410F and by supportive structures connected to the sensing element 440 at the connection region 450A. The connection area 430F of the appendage 400D to the case 410F is sealed to disable leakage of the external fluid medium into the case 410F and is arranged to permit some movement of the connector 420F, such that the connector 420F can transmit the pressure signal from the appendage 400D to the sensing element 440. Sealing the connection area 430F may be carried out by at least one O-ring 815 with an appropriate flexibility placed around the connector 420F. The appendage 400D is shaped to the form of a cap and may partly surround and be in close proximity to the front end of the case. E.g. parts of the cap shaped appendage 400D may be parallel to the front end 810 of the case 410F and further parallel to at least a part of the sides 811 of the case 410F. The appendage 400 may be shaped to have a large area and a high sensitivity to pressure signals in the fluid medium. The form of the appendage 400 may be chosen relating to the direction from which pressure signals are expected.

The appendage 400D is an embodiment of the appendage 400, the case 410F is an embodiment of the case 410. The connector 420F is at least a part of an embodiment of the elongated transmission element 420. The interface 430F arises from an embodiment of the connecting means 430. The connection region 450A arises from an embodiment of the connecting means 450.

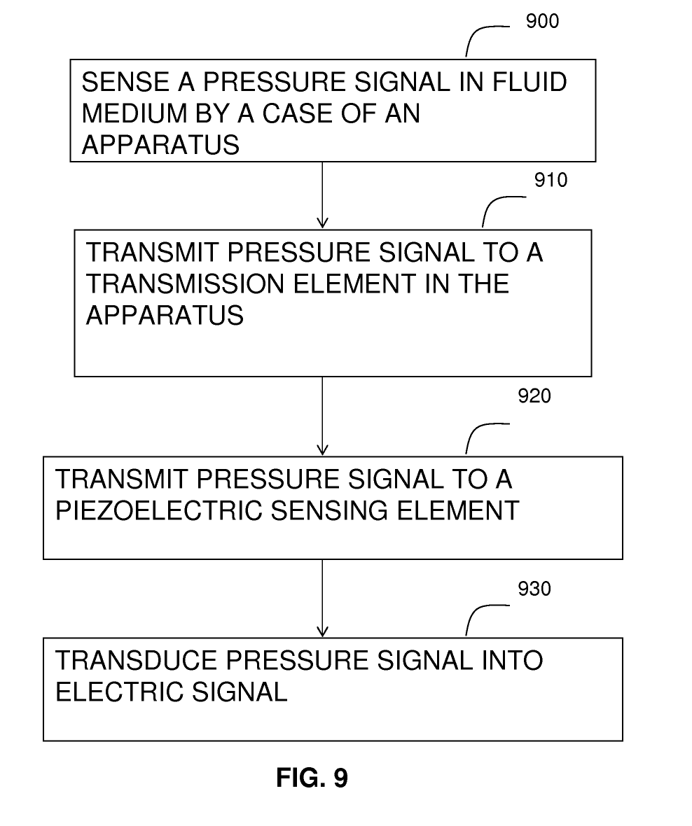

FIG. 9 is a flowchart illustrating a method for measuring pressure signals in a fluid medium, according to some embodiments of the invention. The method comprises the following stages:
Sensing a pressure signal in a fluid medium by a sealed case of a measuring apparatus set in the fluid medium (stage 900). The apparatus may receive the pressure signal on its envelope or at appendages shaped to have a high sensitivity to pressure signals in the fluid medium.
Transmitting the pressure signal from the fluid medium to a transmission element inside the measuring apparatus (stage 910). The apparatus is sealed to protect its inner components from the fluid medium. For example the apparatus may be sealed in a way that allows a long duration of operation or resistivity against chemically aggressive fluid mediums, e.g. with a low pH. The apparatus may be sealed in a way that prevents leakage in a moving fluid medium. The pressure signal is transmitted to the sensing element while maintaining the sealing of the apparatus.
Transmitting the pressure signal from the transmission element to a piezoelectric sensing element (stage 920). The transmission element may amplify the signal, or adjust the intensity of the signal to the sensitivity of the piezoelectric sensing element and to the required resolution of the measurements. The pressure signal is transmitted to the sensing element while maintaining the sealing of the apparatus.
Transducing the pressure signal into an electric signal (stage 930) by the piezoelectric sensing element. The electric signal is then processed and the pressure signal and its components may be analyzed.

According to some embodiments of the invention, the sensing (stage 900) is performed by movements of parts of the case caused by the pressure signal. According to some embodiments of the invention, the sensing (stage 900) is performed by at least one appendage connected to the sealed case.

According to some embodiments of the invention, prior to applying the method, the method parts of the case of the measuring apparatus may be arranged to be moved by pressure signals. According to some embodiments of the invention, prior to applying the method, at least one appendage may be connected to the sealed case. The appendages may improves the sensing of the pressure signal by increasing the surface area in contact with the fluid and controlling the flexibility of different parts of the case and appendages.

According to some embodiments of the invention, sensing the pressure signal (stage 900) and transmitting the pressure signal from the fluid medium to a transmission element and to the sensing element inside the measuring apparatus (stages 910, 920) is carried out while maintaining the measuring apparatus sealed against the fluid medium and avoiding leakage.

According to some embodiments of the invention, the method may result in the amplification of the pressure signal and in adjusting the intensity of the signal as it reaches the sensing element in a way that optimizes the ability to analyze the resulting electric signal, in respect to the required signals.

In the above description, an embodiment is an example or implementation of the inventions. The various appearances of “one embodiment,” “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

It is understood that the phraseology and terminology employed herein is not to be construed as limiting and are for descriptive purpose only.

The principles and uses of the teachings of the present invention may be better understood with reference to the accompanying description, figures and examples.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

The descriptions, examples, methods and materials presented in the claims and the specification are not to be construed as limiting but rather as illustrative only.

Meanings of technical and scientific terms used herein are to be commonly understood as by one of ordinary skill in the art to which the invention belongs, unless otherwise defined.

The present invention can be implemented in the testing or practice with methods and materials equivalent or similar to those described herein.

While the invention has been described with respect to a limited number of embodiments, these should not be construed as limitations on the scope of the invention, but rather as exemplifications of some of the preferred embodiments. Those skilled in the art will envision other possible variations, modifications, and applications that are also within the scope of the invention. Accordingly, the scope of the invention should not be limited by what has thus far been described, but by the appended claims and their legal equivalents.

What is claimed is:

1. A pressure sensor operating in a fluid medium, said sensor comprising:
a sensing element for transducing a pressure signal to an electric signal, said sensing element enclosed within a case;
a mechanical amplifier operatively connected to said sensing element and structured to seal said case and isolate said sensing element from said fluid medium, said mechanical amplifier comprising:
an appendage connected externally to said case and immersed in said fluid medium,
an elongated transmission element operatively connecting said appendage to said sensing element, such that said pressure signal is transmitted from said appendage to said sensing element;
wherein pressure signals in said fluid medium cause movements in said appendage that are transmitted via said elongated transmission element to said sensing element that is isolated from said fluid medium.

2. The pressure sensor of claim 1, wherein said sensing element comprises:
an upper plate connected to said elongated transmission element,
a piezoelectric plate connected to said upper plate,
wherein said upper plate with said elongated transmission element are arranged to amplify the pressure signal to allow an optimal deformation of said piezoelectric plate.

3. The pressure sensor of claim 2, wherein said sensing element further comprising:
a lower metal plate connected to said piezoelectric plate such that at least a portion of said piezoelectric plate is enclosed between the upper plate and the lower metal plate,
at least one upper connection to said upper plate and at least one lower connection to said lower metal plate,
wherein the conductivity of said piezoelectric plate is measured between said upper and lower connections.

4. The pressure sensor of claim 1, wherein said elongated transmission element comprises a pin.

5. The pressure sensor of claim 1, wherein said elongated transmission element comprises a plurality of transmission elements interconnected by joints, wherein said plurality of transmission elements transmit said pressure signal to said sensing element in a range of intensities allowing an optimal analysis of said electric signal.

6. The pressure sensor of claim 1, wherein said appendage is shaped as a plate.

7. The pressure sensor of claim 1, wherein said appendage is shaped to the form of a cap.

8. The pressure sensor of claim 7, wherein said appendage partly surrounding and in close proximity to the front end of said case.

9. The pressure sensor of claim 1, wherein said appendage is shaped as two interconnected parallel plates.

10. The pressure sensor of claim 1, wherein said appendage is an integral part of said case, resulting in sealing said case.

11. The pressure sensor of claim 10, wherein said appendage comprises an inner notch and the distal end of said elongated transmission element is arranged to fit in said notch, such that movements of said appendage are transmitted to said elongated transmission element.

12. The pressure sensor of claim 1, wherein said appendage is connected upon said case by a connector, where said connector is a part of said elongated transmission element, and wherein sealing said case is carried out by at least one O-ring around said connector.

13. A pressure sensor operating in a fluid medium, said sensor comprising:
a sensing element for transducing a pressure signal to an electric signal, said sensing element enclosed within a sealed case;
an elongated transmission element operatively connected to said sensing element and to the inner side of said sealed case, such that said pressure signal is transmitted from the surface of said sealed case to said sensing element;
wherein pressure signals in said fluid medium cause movements in said surface of said sealed case that are transmitted via said elongated transmission element to said sensing element.

14. The pressure sensor of claim 13, wherein the front edge of said sealed case is arranged to transmit the pressure signal to said elongated transmission element.

15. The pressure sensor of claim 14, wherein the front edge of said sealed case is dome shaped.

16. The pressure sensor of claim 14, wherein the front edge of said sealed case has a thin and flexible side walls and a thicker central wall.

17. A method of measuring pressure signals in a fluid medium, said method comprising:
sensing a pressure signal in a fluid medium by a sealed case of a measuring apparatus,
transmitting said pressure signal from said fluid medium to a transmission element inside said measuring apparatus,
transmitting said pressure signal from said transmission element to a piezoelectric sensing element,
transducing said pressure signal into an electric signal by said piezoelectric sensing element.

18. The method of claim 17, further said sensing is performed by movements of parts of said case caused by said pressure signal.

19. The method of claim 17, wherein said sensing is performed by at least one appendage connected to said sealed case.

20. The method of claim 17, wherein said sensing a pressure signal and said transmitting said pressure signal are carried out while maintaining said measuring apparatus sealed against said fluid medium.

21. The method of claim 17, resulting in the amplification of said pressure signal.
